(19) United States
(12) Patent Application Publication
(10) Pub. No.: US 2025/0292168 A1
(43) Pub. Date: Sep. 18, 2025

(54) WORKFORCE OPTIMIZATION SYSTEM

(71) Applicant: Greenworks (Jiangsu) Co. Ltd., Changzhou (CN)

(72) Inventors: Klaus Hahn, Brasselton, GA (US); Nicholas Suchoza, Huntersville, NC (US)

(21) Appl. No.: 18/849,636

(22) PCT Filed: Apr. 6, 2023
(86) PCT No.: PCT/US2023/017673

§ 371 Date: Sep. 23, 2024

Related U.S. Application Data
(60) Provisional application No. 63/328,448, filed on Apr. 7, 2022.

(51) Int. Cl. G06Q 10/0631; A61B 5/0205; G06Q 10/087; G07C 9/00

(52) U.S. Cl. CPC G06Q 10/063114; A61B 5/0205; G06Q 10/087; A61B 2503/20; G07C 9/00896

(57) ABSTRACT

A workforce optimization system includes: a worker tracking device for being carried by individual workers; a tool tracking device for being disposed on a tool, the tool tracking device configured for data communication with the worker tracking device a management device; a backend application in data communication with the worker tracking device, the tool tracking device, and the management device, the backend application programmed to receive information from the worker tracking device and the tool tracking device, process the received information, and transmit the processed information to the management device.

### BACKGROUND OF THE INVENTION

This invention relates generally to workforce optimization and more specifically to methods and apparatus for employee and equipment tracking.

### BACKGROUND

With labor costs being at an all-time high, tracking work time and personnel is vital to ensuring that business operations are as efficient as possible in order to keep costs down. This is especially true in labor-heavy businesses, such as landscaping and grounds maintenance.

Landscape companies have a need to know information such as: the location of their employees; the start time/work time/down time of employees; the location of tools owned by the company; the identity of the worker using company tools; the length of time required to complete a job; the expected time needed to mow one acre or other specified area; Maintenance Alerts; Production Goals; and Trailer Status.

However, one problem with prior art management systems is that this information is collected manually, if at all.

### BRIEF SUMMARY OF THE INVENTION

This problem is addressed by a workforce optimization system which tracks individual employees and pieces of equipment.

According to one aspect of the technology described herein a workforce optimization system includes: a worker tracking device for being carried by individual workers; a tool tracking device for being disposed on a tool, the tool tracking device configured for data communication with the worker tracking device; a management device; a backend application in data communication with the worker tracking device, the tool tracking device, and the management device, the backend application programmed to receive information from the worker tracking device and the tool tracking device, process the received information, and transmit the processed information to the management device.

According to another aspect of the technology described herein, a workforce optimization method includes: providing a worker with a worker tracking device; disposing a tool tracking device on a tool, the tool tracking device configured for data communication with the worker tracking device; providing a management device; coupling a backend software application in data communication with the worker tracking device, the tool tracking device, and the management device; and using the backend software application to receive information from the worker tracking device in the tool tracking device, process the received information, and transmit information to the management device.

### BRIEF DESCRIPTION OF THE DRAWINGS

The invention may be best understood by reference to the following description taken in conjunction with the accompanying drawing figures in which:

FIG. 1 is a diagram of an architecture for a workforce optimization system;

FIG. 2 is a schematic view of a worker tracking device displaying a home screen;

FIG. 3 is a schematic view of a tool and associated battery;

FIG. 4 is a schematic view of a portion of an interior of a vehicle;

FIG. 5 is a schematic diagram of a screen display showing various types of tracking data collected by the system;

FIG. 6 is a schematic view of a worker tracking device displaying a numeric entry keypad;

FIG. 7 is a schematic view of a worker tracking device displaying a live user info menu; and

FIG. 8 is a schematic view of a worker tracking device displaying an emergency alert.

### DETAILED DESCRIPTION OF THE INVENTION

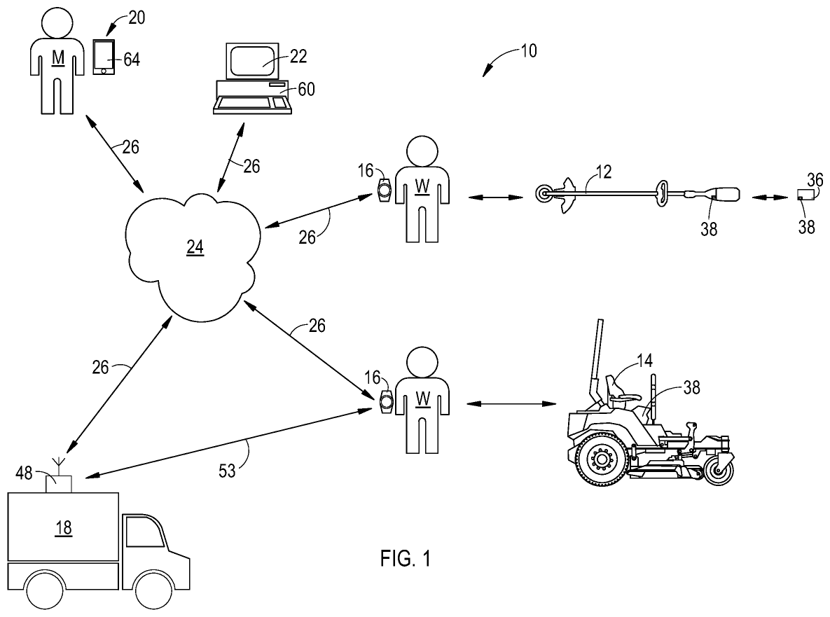

Now, referring to the drawings wherein identical reference numerals denote the same elements throughout the various views, FIG. 1 illustrates an exemplary architecture of a workforce optimization system 10. It includes a plurality of tools, such as the illustrated string trimmer 12 and riding mower 14, a plurality of worker tracking devices 16 each carried by one of a plurality of workers W, a transport vehicle 18, a manager M with a manager device 20, all of which are in data communication with a backend application 22 through a wide-area network 24 such as the Internet, using communications channels 26 such as Wi-Fi or cellular data networks.

Each worker tracking device 16 incorporates one or more processors operable to execute programmed software code as well as at least one transceiver to enable two-way data communication, along with an appropriate power supply, display, and controls as required. The transceiver may incorporate, for example, protocols such as NFC, Bluetooth, Wi-Fi, or Lora. The worker tracking device 16 is portable, i.e., it is of a size and weight enabling it to be practically carried by the worker W continuously when carrying out job duties. Preferably, the worker tracking device 16 is a wearable device. In one example, the worker tracking device 16 may comprise a commercially-available device provisioned with a client software program (also referred to as a “worker application” or “worker app”) containing appropriate programming for carrying out a portion of the method described herein. In one example, the worker tracking device 16 may be a conventional smartphone. In another example, the worker tracking device 16 may be a conventional smartwatch.

The worker tracking device 16 may implement at least one location service, defined as a combination of hardware and software operable to determine the geographic location of the worker tracking device 16. Nonlimiting examples of location services include inertial navigation systems, satellite-based navigation (e.g. GPS, GLONASS, BeiDou, Galileo, IRNSS, or QESS), Wi-Fi-based location, and cellular-based location.

The worker tracking device 16 may incorporate at least one physical sensor, such as a body or air temperature sensor, an atmospheric pressure sensor, or a heart rate sensor. It may also incorporate one or more accelerometers.

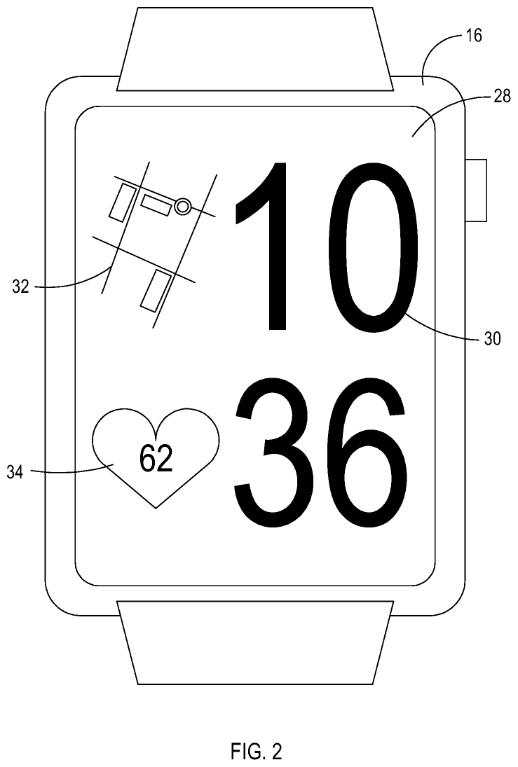

FIG. 2 shows a worker tracking device 16 (integrated into a smartwatch). In this example, the worker tracking device 16 includes a touchscreen 28 which functions as a combination display screen and touch input device. It will be understood that the software running on the worker tracking device 16 is capable of displaying different information and/or menus. In this figure, the touchscreen 28 is displaying a idle screen or lock screen having a time display 30, a location map 32, and a heart rate display 34. The functions of the worker tracking device 16 are described in more detail below.

The system 10 works in conjunction with a plurality of tools. As used herein, the term “tool” refers broadly to any implement, device, or machine that a worker W uses to carry out their assigned work tasks. It is possible to track and control manual implements such as shovels, rakes, hoes, and the like. However, in general an employer will have an interest in tracking and controlling access to power tools such as riding mowers, walk-behind mowers, trimmers, leaf blowers, string trimmers, integers, or power saws.

In the case of electrically-powered tools, the battery pack 36 (FIG. 1) used to power the tools may be considered tools in and of themselves. As used herein, the term “battery pack” is considered to encompass any device suitable for storing and discharging electrical energy. In the illustrated examples, each battery pack 36 is a storage battery including one or more chemical cells, for example lithium ion cells. Other liquid battery chemistries may be substituted, as well as solid state batteries, capacitors, or similar devices which may exist currently or be later developed. The battery pack 36 may include ancillary electrical components such as, transformers, voltage converters, relays, circuit breakers, and/or sensors.

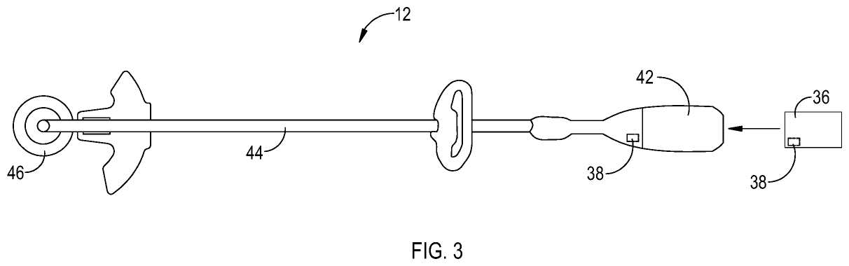

Each tool is provided with a tool tracking device 38. Each tool tracking device 38 incorporates one or more processors operable to execute programmed software code as well as at least one transceiver to enable two way data communication, along with an appropriate power supply, display, and controls as required. The tool tracking device 38 may be configured to communicate over a short-range data protocol such as Wi-Fi, Bluetooth, or Bluetooth low energy (BLE). Additionally or alternatively, the tool tracking device 38 may incorporate a longer-range data protocol such as cellular (e.g. 4G, 5G networks). In one example embodiment, the tool tracking device 38 is configured for two-way wireless data communication with a worker tracking device 16 and/or with the vehicle 18. The tool tracking device 38 may be attached to the tool or incorporated internally. FIG. 3 illustrates an example tool in the form of a string trimmer 12 having a power head 42 with a removable, rechargeable battery pack 36, a shaft 44, and a rotating trimmer head 46. A first tool tracking device 38 is located internal to the power head 42, as shown schematically in this figure. A second tool tracking device 38 is located internal to the removable battery pack 36, shown schematically.

Optionally, the tool tracking device 38 may implement at least one location service, defined as a combination of hardware and software operable to determine the geographic location of the tool tracking device 38. Nonlimiting examples of location services include inertial navigation systems, satellite-based navigation (e.g. GPS, GLONASS, BeiDou, Galileo, IRNSS, or QESS), Wi-Fi-based location, and cellular-based location. Alternatively, the tool tracking device 38 need not contain an independent location service. Instead, the location of the tool tracking device 38 may be determined by triangulation, trilateration, and/or localization, based on the tool tracking device 38 being in communication (and thus in relatively close proximity to) with one or more devices that are in longer-range communication, such as the worker tracking devices 16, or vehicle tracking devices 48.

The system 10 may work in conjunction with one or more vehicles 18. Such vehicles 18 are typically used to transport workers between company facilities and jobsites, and to store and transport tools for the workers W. The vehicle 18 may be self-propelled (e.g., a landscaping truck), or it may be a towed vehicle (e.g., a landscaping trailer).

Each vehicle 18 is provided with a vehicle tracking device 48. Each vehicle tracking device 48 incorporates one or more processors operable to execute programmed software code as well as at least one transceiver to enable two way data communication, along with an appropriate power supply, display, and controls as required. For example, it may include a short range transceiver such as Wi-Fi or Bluetooth, or a longer-range protocol such as a cellular transceiver (e.g. 4G, 5G network protocol).

The vehicle tracking device 48 may implement at least one location service, defined as a combination of hardware and software operable to determine the geographic location of the vehicle tracking device 48. Nonlimiting examples of location services include inertial navigation systems, satellite-based navigation (e.g. GPS, GLONASS, BeiDou, Galileo, IRNSS, or QESS), Wi-Fi-based location, and cellular-based location.

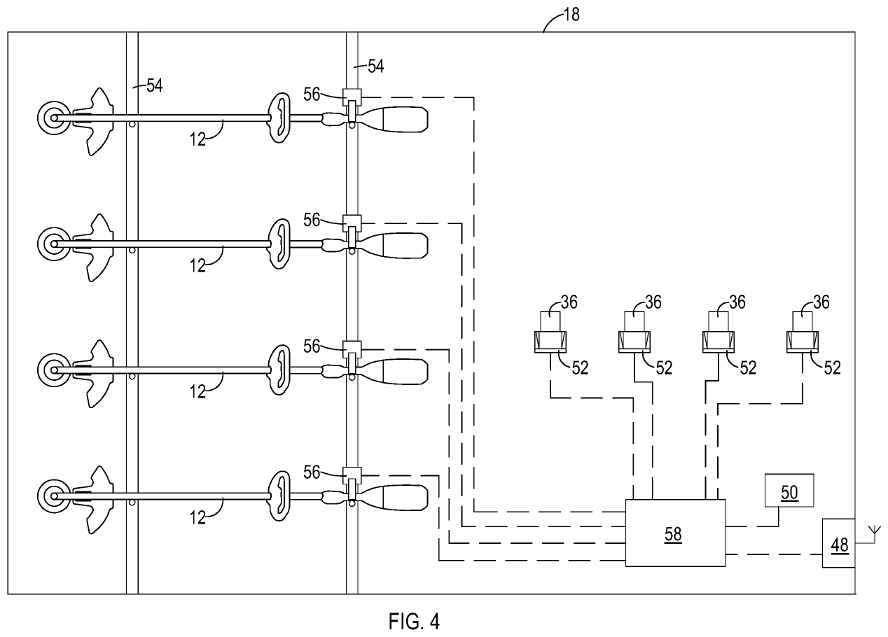

The vehicle 18 is outfitted to store and/or charge one or more tools. FIG. 4 shows a schematic view of a portion of the interior of a vehicle 18. It includes a charging power source 50 which provides electrical power for charging one or more individual battery packs 36 or tools that include batteries. Examples of suitable charging power sources include storage batteries and portable generators.

In the illustrated example the vehicle interior includes a plurality of chargers 52 which are configured to receive individual battery packs 36 and charge those battery packs 36 using electrical power from the charging power source 50.

In the illustrated example the vehicle interior includes tool storage racks 54 which carry a plurality of tools. In this example, the tools are cordless battery-powered string trimmers 12. Each tool storage rack 54 includes electrically-operated tool latches 56. Each tool latch 56 is movable between an open or unlocked position in which the tool can be inserted or removed from the tool storage rack 54, and a closed or locked position in which the tool is secured to the tool storage rack 54.

A main controller 58 in the vehicle interior is operably coupled to the charging power source 50, the chargers 52, and the charging power source 50. incorporates one or more processors operable to execute programmed software code. It is coupled to the vehicle tracking device 48 to enable two-way data communication.

Using data communicated from the worker tracking devices 18 and tool tracking devices 38, the vehicle 18 (specifically main controller 58) will be able to track who enters the vehicle 18 and which tools are taken from the vehicle 18 or brought back to the vehicle 18. This will provide accounting and tracking of who takes a tool. When the vehicle 18 comes back to a shop or central service location, the manager M will be able to review this information and know exactly what is on the vehicle 18 and if there are tools that need to be serviced.

The system 10 includes a backend application 22 which communicates with the other system components through the wide-area network 24. It will be understood that the backend application 22 may be hosted on one or more servers or individual user devices, which are connected to the wide-area network 24. A single server 60 hosting the backend application 22 is depicted schematically in FIG. 1.

The system 10 includes at least one management device 20 for use by manager M. It communicates with the backend application 22 through the wide-area network 24. It will be understood that the management device 20 may be embodied as a conventional desktop computer or a mobile computing device (e.g. smart phone) programmed with appropriate software. A smartphone 64 hosting a management app is depicted schematically in FIG. 1.

The components described above may be used to provide various workforce optimization functions. Examples of these functions are described below.

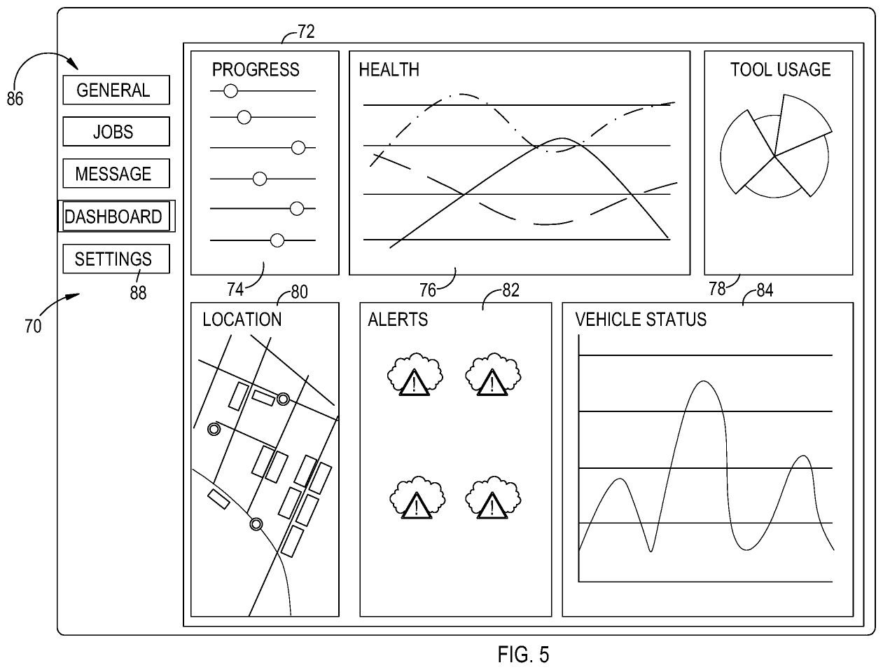

The system 10 collects data as it is being used. This permits full data visibility of tool data, employee markers, and health data. The system 10 may be programmed collect, analyze, and manipulate data and produce output formatted for display on the management device 20 or other suitable device. FIG. 5 illustrates a screen display 70 formatted as a “dashboard” 72 which displays top-level summary data from various sources. In the illustrated example, the dashboard 72 includes a job progress display 74, an employee health tracking chart 76, a tool usage pie chart 78, a location display 80 with employee and tool locations superimposed on a map, a summary of alerts 82, and a vehicle status tracking chart 84. The screen display 70 also includes a user menu 86 with interactive icons 88 for interacting with the dashboard activating various functions.

As part of the data visibility function, the system 10 may be programmed to display an “employee dashboard” on the management device 20 which permits viewing of the status of the employees as reported by the worker tracking devices 16. More specifically, the backend application 22 may be programmed to collect, analyze, and manipulate data and produce output formatted for display on the management device 20. For example, the employee dashboard may be of a similar format to the main dashboard 72 shown in FIG. 5 and may display text, graphics, icons, or other information showing the location of each worker W, the movement of the worker W superimposed on a map, and/or a log of the worker's movements over a given period of time.

As another example, the employee dashboard may display icons or other information showing what tools a specific worker W has used, in what locations, and during what period of time.

As another example, the employee dashboard may display icons or other information showing health data for a worker W. Examples of health data include heart rate and/or blood pressure, blood oxygen monitoring, or movement data such as the number of steps are distance the employee has traveled. They may also be capable of detecting if a worker has fallen, based on signals from the accelerometer described above.

The system may be programmed to display an “asset dashboard” on the management device 20 which permits viewing of the status of the tools and/or vehicles 18 as reported by the worker tracking devices 16 and/or vehicle tracking devices 48. More specifically, the backend application 22 may be programmed to collect, analyze, and manipulate data and produce output formatted for display on the management device 20. For example, the asset dashboard may be of a similar format to the main dashboard 72 shown in FIG. 5 and may display icons or other information showing the location of each tool or vehicle 18, the movement of the tool or vehicle 18 superimposed on a map, and/or a log of the tool or vehicle movements over a given period of time. In the case of the worker tracking devices 18, it is noted that these may communicate information to the backend application 22 directly over the wide-area network 24. Alternatively, if they are not provided with a cellular or other long-range data connection protocol, the worker tracking devices 16 may store the data they collect and then transfer that data to another device acting as a hub when in sufficiently close proximity. For example, one of the tool tracking devices 38 or the vehicle tracking device 48 (provided with a long-range communications protocol) may act as a hub. In this option, the worker tracking device 16 would transfer the stored data using a short-range protocol such as NFC, Bluetooth, or Lora (see link 53 in FIG. 1) The hub would then transfer the information to the side-area network 24

For the vehicle 18, the asset dashboard may display the location of the vehicle 18 as well as an inventory of the tools that are currently on the vehicle 18, the status of the charging power source 50 (e.g. fuel level or battery charge level), the status of any battery packs 36 that are being charged by the chargers 52, and the time remaining to charge the batteries.

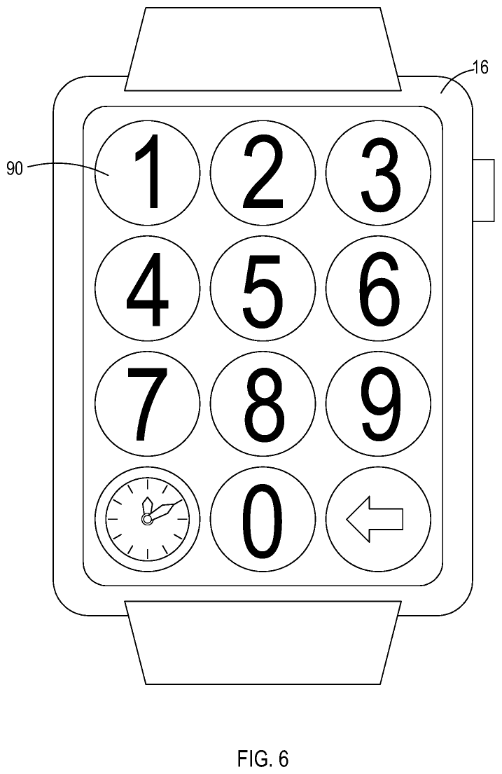

The system 10 may provide for timekeeping and job tracking. For example, all of the worker tracking devices 16 may be placed in a charging station at a central office (not shown). Each worker W is assigned a unique ID number. When the worker W comes to the central office to begin their workday, they secure a worker tracking device 16 and enter the ID number. FIG. 6 shows a worker tracking device 16 displaying a numeric keypad 90 for this purpose. This information is collected by the backend application 22m so that the manager M knows the worker W has shown up for the day. The system 10 may be programmed such that the worker W is not paid and does not have permission to use the tools if the worker tracking device 16 is not used.

The system 10 may provide for a tool checkout function. Referring to FIG. 4, the tool latches 56 of the tool racks 54 may be maintained in the locked position condition. When a worker W wishes to use the tool, they may unlock the tool latch 56 by bringing their worker tracking device 16 near the latch 56 and/or by activating a specific menu option on the worker tracking device 16. This causes the main controller 58 to unlock the appropriate tool latch 56, permitting the worker W to access the tool. This also begins tracking of the tool until the worker W replaces it on the tool rack 54.

A similar process may be used to control access to larger tools such as riding mowers 14. In this case, the tool tracking device 38 would render the operational parts of the tool inoperable, for example by electrically disconnecting the internal battery packs. When a worker wishes to use the tool, they may unlock the tool by bringing their worker tracking device 16 near the tool tracking device 38 and/or by activating a specific menu option on the worker tracking device 16. This causes the tool tracking device 38 to render the tool usable, for example by electrically connecting the internal battery packs.

A similar process may be used to control access to the vehicle 18. In this case, the main controller 58 would render the operational parts of the vehicle 18 inoperable, for example by electrically disconnecting the internal battery packs. Alternatively, the controller 58 may be used to maintain one or more electromechanical locks (for example door locks) of the vehicle 18 in a locked condition. When a worker wishes to access the vehicle 18, they may unlock it by bringing their worker tracking device 16 near the main controller 58 or an appropriate receiver (not shown) mounted on the vehicle 18, and/or by activating a specific menu option on the worker tracking device 16. This causes the main controller 58 to render the vehicle usable, for example by electrically connecting the internal battery packs, or by unlocking the electromechanical lock to provide physical access to the vehicle 18.

The system 10 may provide for two-way communication between the management device 20 and the worker tracking devices 16. This may take the form of text messaging, voice calls, and/or video calls.

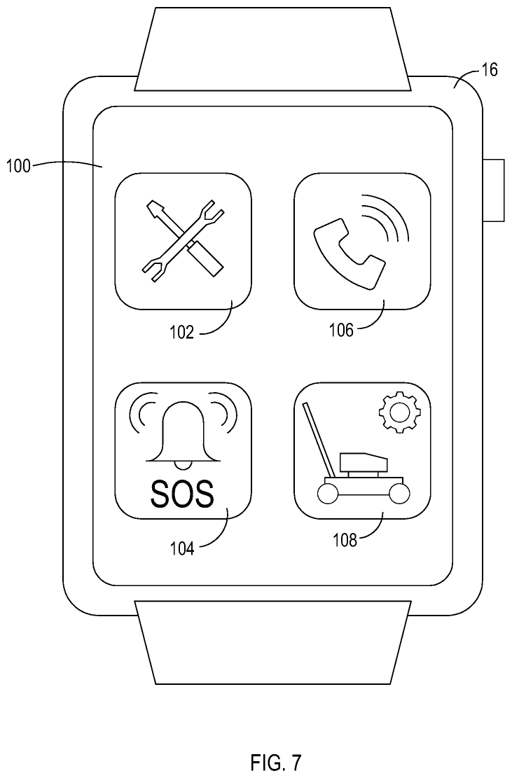

The system 10 may be programmed to provide a live user info function. FIG. 7 illustrates a worker tracking device 16 displaying a worker assistance menu 100. It includes a tool failure/service button 102. This is programmed to place a worker W in communication with a service department or a database of self-help instructions. It also includes a SOS emergency call button 104. This is programmed to place a worker W in communication with emergency services such as police or fire squad. It includes a call foreman/headquarters button 106. This is programmed to place a worker in communication with a manager M or other designated person. Finally, it includes a tool information button 108. This is programmed to display the status of the tool being used, such as remaining battery life.

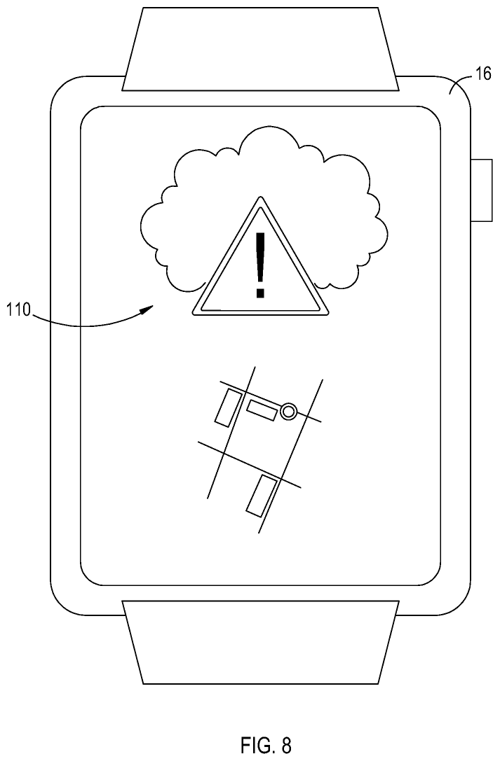

The system may provide for emergency alerts. These may take the form of audible and/or visible alerts displayed on the worker tracking devices 16. FIG. 8 illustrates a worker tracking device 16 displaying an emergency weather alert 110. For example, the backend application 22 may access public weather data sources on the Internet and/or government-provided Internet services. It may receive and relay emergency alerts for nearby or expected severe weather such as storms, floods, or tornadoes. It may also receive and relay alerts for inclement weather such as low temperature or rain. These conditions may not represent a direct safety threat, but may make work impractical or inconvenient.

Using the system described herein, managers and company owners can monitor their personnel at anytime to understand the work that is being accomplished without ever having to be on the jobsite. They can directly communicate with the worker at any time for status updates on a job or the worker can communicate with them for any needs they have in the field. They can tie the information to personnel evaluations, incentive programs, billing to customers, tool use and maintenance schedules. This will increase efficiency of personnel, potentially improve their health and safety, lead to more jobs being completed, and make allocating personnel easier than ever.

The foregoing has described a workforce optimization system. All of the features disclosed in this specification, and/or all of the steps of any method or process so disclosed, may be combined in any combination, except combinations where at least some of such features and/or steps are mutually exclusive.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends, or to any novel one, or any novel combination, of the steps of any method or process so disclosed.

What is claimed is:

1. A workforce optimization system, comprising:
a worker tracking device for being carried by individual workers;
a tool tracking device for being disposed on a tool, the tool tracking device configured for data communication with the worker tracking device;
a management device;
a backend application in data communication with the worker tracking device, the tool tracking device, and the management device, the backend application programmed to receive information from the worker tracking device and the tool tracking device, process the received information, and transmit the processed information to the management device.

2. The workforce optimization system of claim 1, wherein the backend application is programmed to generate a dashboard displaying status of at least one of the worker and the tool and transmit the dashboard to the management device for display.

3. The workforce optimization system of claim 1, wherein the backend application is programmed to permit or deny the worker access to the tool based on predetermined criteria.

4. The workforce optimization system of claim 1, wherein the backend application is programmed to track at least one of a location of the worker and usage of the tool by a worker in response to signals from the worker tracking device and the tool tracking device.

5. The workforce optimization system of claim 1, wherein the backend application is programmed to track at least one health parameter of the worker in response to signals from the worker tracking device.

6. The workforce optimization system of claim 1, further comprising a vehicle configured for storage of the tool, the vehicle including a vehicle tracking device in data communication with the backend application.

7. The workforce optimization system of claim 6, wherein the vehicle tracking device is programmed to track the presence of the tool tracking device within the vehicle.

8. The workforce optimization system of claim 6, wherein the vehicle includes a tool latch movable between a locked position and a unlocked position, wherein the backend application is programmed to lock or unlock the tool latch in response to signals from the worker tracking device.

9. The workforce optimization system of claim 6, wherein the vehicle includes a charging power source coupled to a charger for a battery pack.

10. The workforce optimization system of claim 1, wherein the backend application is programmed to track job information in response to a signal from the worker tracking device.

11. A workforce optimization method, comprising:
providing a worker with a worker tracking device;
disposing a tool tracking device on a tool, the tool tracking device configured for data communication with the worker tracking device
providing a management device;
coupling a backend software application in data communication with the worker tracking device, the tool tracking device, and the management device; and
using the backend software application to receive information from the worker tracking device in the tool tracking device, process the received information, and transmit information to the management device.

12. The method of claim 11, further comprising using the backend application is to generate a dashboard displaying status of at least one of the worker and the tool and transmit the dashboard to the management device for display.

13. The method of claim 11, further comprising using the backend application to permit or deny the worker access to the tool based on predetermined criteria.

14. The method of claim 11, further comprising:
providing a vehicle configured for storage of the tool, the vehicle including a vehicle tracking device in data communication with the backend application; and
is the backend application to track the location of the vehicle based on signals from the vehicle tracking device.

15. The method of claim 14, further comprising using the vehicle tracking device to track the presence of the tool tracking device within the vehicle.
